(19) United States
(12) Patent Application Publication
(10) Pub. No.: US 2024/0041738 A1
(43) Pub. Date: Feb. 8, 2024

(54) TWO-COMPONENT PROCESS FOR COATING KERATIN MATERIALS WHICH CONSISTS IN APPLYING TO SAID MATERIALS A COMPOSITION WITH A POLYPHENOL AND A COMPOSITION WITH A COMPOUND THAT IS CAPABLE OF FORMING HYDROGEN BONDING WITH SAID POLYPHENOL

(71) Applicant: L'OREAL, Paris (FR)

(72) Inventors: Philippe ILEKTI, Chevilly Larue (FR); Grégory PLOS, Chevilly Larue (FR)

(21) Appl. No.: 18/256,734

(22) PCT Filed: Dec. 3, 2021
(86) PCT No.: PCT/EP2021/084210

§ 371 Date: Jun. 9, 2023

(30) Foreign Application Priority Data
Dec. 15, 2020 (FR) FR2013285

(51) Int. Cl. A61K 8/49; A61K 8/60; A61Q 1/02

(52) U.S. Cl. CPC A61K 8/498; A61K 8/602; A61Q 1/02; A61K 2800/31; A61K 2800/884; A61K 2800/43

(57) ABSTRACT

The present invention relates to a cosmetic process for coating keratin materials, notably for care and/or makeup, more particularly for makeup, which consists in applying to said keratin materials:
a) at least one composition (A) comprising, notably in a physiologically acceptable medium, at least one polyphenol X comprising at least two different phenol groups; and
b) at least one composition (B) comprising, notably in a physiologically acceptable medium, at least one compound Y including at least two functional groups Gy, which may be identical or different, which are capable of forming at least two hydrogen bonds with said phenol groups of the polyphenol X; said compositions (A) and (B) being applied to the keratin materials
i) simultaneously; or ii) in the form of an extemporaneous mixture at the time of use; or iii) successively, irrespective of the order of application.

### TECHNICAL FIELD

The present invention relates to the field of coating keratin materials, notably the field of care and/or makeup, and is directed towards proposing a process for coating keratin materials, notably for care and/or makeup, which consists in applying to said materials a composition (A) comprising at least one polyphenol X comprising at least two different phenol groups and a composition (B) comprising at least one compound Y comprising at least two functional groups Gy, which may be identical or different, which are capable of forming at least two hydrogen bonds with said phenol groups of said polyphenol X.

At the present time on the market for caring for and making up keratin materials, many products claim persistence throughout the day, withstanding external factors such as water, sebum, mechanical friction, etc. (waterproof mascara, food-proof lipsticks, long-lasting foundations). Long-lasting products for the lips, the eyelashes, the eyebrows or the face, which can be used at home, are mainly based on synthetic coating polymers in the presence of organic solvents. For making up the lips and compositions for making up the face, compositions comprising a silicone resin as coating agent are known, such as the compound having the INCI name: Trimethylsiloxysilicate or a compound having the INCI name: Polypropylsilsesquioxane, or alternatively a silicone acrylate copolymer such as the product having the INCI name: Acrylates/polytrimethylsiloxymethacrylate copolymer). Long-lasting makeup products for the eyelashes and/or the eyebrows (mascaras, eyeliners) use, for their part, waxes or film-forming polymer particles in aqueous suspension of the latex type (i.e.: styrene/acrylate copolymers).

Besides these long-lasting products, the current trend is towards semi-permanent makeup. Specifically, in recent years, conventional makeup products have met with competition from the market of semi-permanent makeup in professional salons. It is encountered in the sector of makeup for the eyes (semi-permanent mascara, permanent eyelash makeup, eyelash extensions, etc.), for the eyebrows (semi-pigmentation known as micro-blading), for the complexion (freckles, beauty spots or the whole face, glowing or healthy-complexion effect) or for the lips (semi-permanent tattooing). This new trend is driving consumers towards seeking increasingly long persistence for greater practicality (avoiding having to apply and remove makeup daily, healthy complexion effect immediately on waking up, etc.).

However, consumers, who are increasingly demanding as regards the composition of their cosmetic products, are also seeking to use products with ingredients that are well tolerated such as natural ingredients, with ingredients which have little or no environmental impact and/or ingredients which are compatible with numerous packagings.

The aim of the present invention is to propose compositions which offer excellent persistence of the expected cosmetic effects, notably the colour of the makeup on keratin materials (skin, lips, nails, hair, eyelashes, eyebrows) which may extend from one day, including makeup removal at the end of the day, to persistence over several days, which is resistant to mechanical friction, water, sweat and perspiration, sebum, oil, cleansing products such as shower gels, shampoos, two-phase products and certain micellar waters.

In addition, the aim of the present invention is to propose compositions which afford persistence of the expected cosmetic effects, notably the colour of the makeup on keratin materials, combined with a good level of comfort in comparison with conventional systems, in particular based on silicone resin. The term “comfort” means absence of tackiness.

In the course of its studies, the Applicant has discovered, unexpectedly, that the objectives as defined previously were achieved with a cosmetic process for coating keratin materials, notably for care and/or makeup, more particularly makeup, which consists in applying to said keratin materials:
a) at least one composition (A) comprising, notably in a physiologically acceptable medium, at least one polyphenol X comprising at least two different phenol groups; and
b) at least one composition (B) comprising, notably in a physiologically acceptable medium, at least one compound Y including at least two functional groups Gy, which may be identical or different, which are capable of forming at least two hydrogen bonds with said phenol groups of the polyphenol X;
said compositions (A) and (B) being applied to the keratin materials i) simultaneously; or ii) in the form of an extemporaneous mixture at the time of use; or iii) successively, irrespective of the order of application.

The inventors have found, unexpectedly, that the coating agent deposited on the upper layers of the keratin materials and resulting from the hydrogen bonding interaction, at room temperature and atmospheric pressure, of at least one polyphenol X comprising at least two different phenol groups with at least one compound Y including at least two functional groups, which may be identical or different, which are capable of forming at least two hydrogen bonds with said phenol groups of the polyphenol X, allowed excellent persistence of the expected cosmetic effects on the keratin materials (skin, lips, nails, hair, eyelashes, eyebrows) which may extend from one day, including makeup removal at the end of the day, to persistence over several days, which is resistant to mechanical friction, water, sebum, oil, cleansing products such as shower gels, shampoos, two-phase products and certain micellar waters.

The inventors have also found that the coating agent obtained according to the invention made it possible to obtain a good level of comfort and notably the absence of a tacky effect.

Furthermore, said coating agent may be obtained with natural compounds or compounds of natural origin which are capable of forming hydrogen bonding with said polyphenols.

This discovery forms the basis of the invention.

### Subjects of the Invention

Thus, according to one of its aspects, the present invention relates to a cosmetic process for coating keratin materials, notably for care and/or makeup, more particularly for makeup, which consists in applying to said keratin materials:
a) at least one composition (A) comprising, notably in a physiologically acceptable medium, at least one polyphenol X comprising at least two different phenol groups; and
b) at least one composition (B) comprising, notably in a physiologically acceptable medium, at least one compound Y including at least two functional groups Gy, which may be identical or different, which are capable of forming at least two hydrogen bonds with said phenol groups of the polyphenol X;
said compositions (A) and (B) being applied to the keratin materials
i) simultaneously; or ii) in the form of an extemporaneous mixture at the time of use; or iii) successively, irrespective of the order of application.

Another subject of the present invention is a cosmetic kit for coating keratin materials, notably for care and/or makeup, comprising at least:
a) a first composition (A) as defined previously; and
b) a second composition (B) as defined previously; said compositions (A) and (B) being packaged separately.

### Definitions

In the context of the present invention, the term “keratin material” notably means the skin such as the face, the body, the hands, the area around the eyes, the lips, keratin fibers such as head hair, the eyelashes, the eyebrows, bodily hair and the nails. For the purposes of the present invention, this term “keratin materials” also extends to synthetic false eyelashes and false eyebrows, and false nails.

The term “physiologically acceptable” means compatible with the skin and/or its integuments, which has a pleasant colour, odour and feel, and which does not cause any unacceptable discomfort (stinging or tautness) liable to discourage the consumer from using this composition.

For the purposes of the invention, the term “hydrogen bonding interaction” means an interaction involving a hydrogen atom of one of the two reagents and an electronegative heteroatom of the other reagent, such as oxygen, nitrogen, sulfur and fluorine. In the context of the invention, the hydrogen bonding is formed between the reactive hydroxyl functions (OH) of the phenol groups of the polyphenol X and the functional groups Gy of the compound Y containing these electronegative heteroatoms and which are capable of forming hydrogen bonding with said phenol groups of the polyphenol X.

By “coating agent formed by interaction by hydrogen bonds of at least one polyphenol X comprising at least two different phenol groups with the compound Y” means that the conditions are met so that the reaction can be carried out between the two reagents, in particular that
i) the amount of polyphenol X is sufficient in the composition containing it, and
ii) the compound Y is soluble, miscible or solubilized by another solvent in the medium of the composition containing it, and
iii) the compound Y has a sufficient number of hydrogen bond acceptor groups to react with the phenol groups of polyphenol X and, in the medium of the composition containing it, and
iv) the compound Y, in the medium of the composition containing it, does not comprise in its structure any group which does not allow the formation of hydrogen bonding with the functions of the reactive phenol groups of the polyphenol X, such as for example, one or more anionic groups.

The term “room temperature” means 250° C.

The term “atmospheric pressure” means 760 mmHg, i.e. 10′ pascals.

The term “natural compound” refers to any compound derived directly from a plant without having undergone any chemical modification.

The term “synthetic compound” refers to any compound which is neither naturally existing nor a derivative of a compound of natural origin.

The term “compound of natural origin” refers to any compound obtained from a plant, which has undergone one or more chemical modifications, for example by organic synthesis reaction, without the properties of the natural compound having been modified.

The term “coating agent” refers to any compound which is capable of forming a deposit on the surface of keratin materials so as to cover them.

### Polyphenol X

The polyphenols that may be used according to the present invention include in their structure at least two different phenol groups.

The term “polyphenol” refers to any compound containing in its chemical structure at least two and preferably at least three phenol groups.

The term “phenol group” refers to any group comprising an aromatic ring, preferably a benzene ring, including at least one hydroxyl group (OH).

The term “different phenol groups” refers to phenol groups that are chemically different.

The polyphenols X that may be used according to the invention may be synthetic or natural. They may be in isolated form or contained in a mixture, notably contained in a plant extract. Polyphenols are phenols comprising at least two phenol groups that are differently substituted on the aromatic ring.

The two classes of polyphenols are flavonoids and non-flavonoids.

Examples of flavonoids that may be mentioned include chalcones such as phloretin, phloridzin, aspalathin or neohesperidin; flavanols such as catechin, fisetin, kaempferol, myricetin, (CI uercetin, rutin, procyanidins, proanthocyanidins, pyroanthocyanidins, theaflavins or thearubigins (or thearubrins); dihydroflavonols such as astilbin, dihydroquercetin (taxifolin) or silibinin; flavanones such as hesperidin, neohesperidin, hesperetin, naringenin or naringin; anthocyanins such as cyanidin, delphinidin, malvidin, peonidin or petunidin; catechin tannins such as tannic acid; isoflavonoids such as daidzein or genistein; neoflavanoids; lignans such as pyroresorcinol; and mixtures thereof.

Among the natural polyphenols that may be used according to the invention, mention may also be made of lignins

Examples of non-flavonoids that may be mentioned include curcuminoids such as curcumin or tetrahydrocurcumin; stilbenoids such as astringin, resveratrol or rhaponticin; aurones such as aureusidin; and mixtures thereof.

As polyphenols that may be used according to the invention, mention may also be made of chlorogenic acid, verbascoside; coumarins substituted with phenols.

According to a particular embodiment of the invention, the polyphenol X will be chosen from catechin tannins such as gallotannins chosen from tannic acid; ellagitannins such as epigallocatechin, epigallocatechin gallate, castalagin, vescalagin, vescalin, castalin, casuarictin, castanopsinins, excoecarianins, grandinin, gradinin, roburins, pterocarinin, acutissimin, tellimagrandins, sanguiin, potentillin, pedunculagin, geraniin, chebulagic acid, repandisinic acid, ascorgeraniin, stachyurin, casuarinin, casuariin, punicacortein, coriariin, cameliatannin, isodeshydrodigalloyl, dehydrodigalloyl, hellinoyl, punicalagin and rhoipteleanins.

According to a particular embodiment of the invention, the polyphenol X is epigallocatechin.

According to a particular embodiment of the invention, the polyphenol X is a green tea extract having the INCI name Green Tea Extract, notably comprising at least 45% epigallocatechin relative to the total weight of said extract, for instance the commercial product sold under the name Dermofeel Phenon 90 M-C® sold by the company Evonik Nutrition & Care or the commercial product sold under the name Tea Polyphenols Green Tea Extract® by the company Tayo Green Power.

According to a particular embodiment of the invention, the polyphenol X is a procyanidin or a mixture of procyanidins, in particular an extract of maritime pine bark having the INCI name Pinus pinaster Bark/Bud Extract, notably comprising at least 65% by weight of procyanidins relative to the total weight of said extract, such as the commercial product sold under the name Pycnogenol® sold by the company Biolandes Arômes.

Tannic acid will be used more particularly as polyphenol X.

According to a particular embodiment, the polyphenol (s) X according to the invention will be present in a content equal or greater than 0.8% by weight, preferably equal or greater than 1.0% by weight, more particularly equal or greater than 2.0% by weight relative to the total weight of the composition (A).

According to a particular embodiment, the polyphenol (s) X according to the invention will be present in a content from 1,0 to 30.0% by weight and more particularly of from 2.0 to 30% relative to the total weight of the composition (A).

### Compound Y

The compounds Y that may be used according to the invention comprise in their chemical structure at least two functional groups Gy, which may be identical or different, which are capable of forming at least two hydrogen bonds with the phenol groups of the polyphenol X comprising at least two different phenols.

The compounds Y that may be used according to the invention comprise at least two functional groups, which may be identical or different, chosen from hydroxyl (OH), acid anhydride (R—CO—O—CO—R), ether (R1-O—R2), amino (NHR1R2R3), amide (ROCNR′R″), carbamate, urethane (R—HN—(C═O) O—R′), carbamide, urea (CO(NH₂)₂), thiol (RSH), glyceryl, acrylate, acrylamide, vinylpyrrolidone, vinyl alcohol, vinylamine, vinylformamide, and mixtures thereof.

In a preferred embodiment, the molar mass of compound Y is greater than 200 g/mol, or even greater than 350 g/mol.

As examples of compounds Y that are capable of reacting with polyphenols X such as those indicated previously, mention may be made of:

According to a particular embodiment, the compound(s) Y, in the medium of the composition containing it (them), does (do) not contain any anionic group in its(their) structure.

According to a particular embodiment, the compound(s) Y in accordance with the invention will be different from the sugars obtained from fruits or vegetables, in particular simple sugars issued from apple extracts such as glucose, sucrose, saccharose, fructose and sorbitol.

As examples of compounds Y that are capable of reacting with polyphenols X such as those indicated previously, mention may be made of:
(1) Glycerolated alkyl ethers such as glyceryl lauryl ether.
(2) Modified or unmodified polysaccharides, which are preferably nonionic. The polysaccharides that are suitable for use in the invention may be homopolysaccharides such as fructans, glucans, galactans and mannans or heteropolysaccharides such as hemicellulose. They may be starchy polysaccharides such as native or modified starches. The non-starchy polysaccharides may be chosen from polysaccharides produced by microorganisms; polysaccharides isolated from algae, and higher plant polysaccharides, such as homogeneous polysaccharides, in particular celluloses and derivatives thereof or fructoses, heterogeneous polysaccharides such as galactomannans, glucomannans and pectins, and derivatives thereof; and mixtures thereof. In particular, the polysaccharides may be chosen from fructans, glucans, amylose, amylopectin, glycogen, pullulan, dextrans, celluloses and derivatives thereof, in particular methylcelluloses, hydroxyalkylcelluloses and ethylhydroxyethylcelluloses, cetylhydroxyethylcelluloses, mannans, xylans, arabans, galactans, galacturonans, chitin, chitosans, glucuronoxylans, arabinoxylans, xyloglucans, glucomannans, arabinogalactans, agars, karaya gums (about 40% acid), locust bean gums, guar gums and nonionic derivatives thereof, in particular hydroxypropyl guar, and biopolysaccharide gums of microbial origin, in particular scleroglucan gums. They are notably chosen from celluloses such as cetylhydroxyethylcelluloses; guar gums that are notably modified such as hydroxypropyl guars, agarose; pullulans, inulins and starches.
(3) Polyglycerolated alkyl ether nonionic surfactants, in particular chosen from polyglyceryl-2 oleyl ether and polyglyceryl-4 oleyl ether.
(4) Glycerol or polyglycerol esters of fatty acids, which are optionally polyhydroxylated, in particular chosen from polyglyceryl-3 polyricinoleate, polyglyceryl-2 diisostearate, polyglyceryl-4 diisostearate, polyglyceryl-4 caprate, polyglyceryl-2 stearate, polyglyceryl-3 dicitrate/stearate, polyglyceryl-10 dioleate, polyglyceryl-3 diisostearate, polyglyceryl-2 triisostearate, polyglyceryl-10 laurate, glyceryl stearate citrate and polyglyceryl-2 dipolyhydroxystearate.
(5) Polyoxyethylenated or polyglycerolated waxes, notably chosen from polyoxyethylenated ester waxes such as polyoxyethylenated (120 OE) jojoba wax (INCI name: Jojoba Wax PEG-120 Esters), PEG-8 Beeswax, PEG-60 Lanolin, PEG-75 Lanolin, PPG-12-PEG-50 Lanolin and Polyglyceryl-3 Beeswax.
(6) Polyethylene glycols of the type HO—CH₂—CH₂)ₙ—OH
in particular chosen from PEG-6, PEG-8, PEG-14M, PEG-20, PEG-45M, PEG-90, PEG-90M, PEG-150, PEG-180 and PEG-220.
(7) Poloxamers of the type HO—(CH2-CH2-O)n-(CHCH3-CH2-O)O—(CH₂—CH₂—O)ₚ—H, in particular chosen from Poloxamer 124®, Poloxamer 184®, Poloxamer 338© and Poloxamer 338®.
(8) Polypropylene glycol alkyl ethers of the type: CₙH₂ₙ₊₁—(O—C(CH₃)H—CH₂)ₒ—(O—CH₂—CH₂)P—OH
in particular chosen from PPG-26-Buteth-26, PPG-5-Ceteth-20 and PPG-6-Decyltetradeceth-30.
(9) Compounds of the type:
H(O—C(CₙH₂ₙ₊₁)—CH₂)ₒ—(CH₂—CH₂—O)ₚ—(CH₂—C(CH₂—C(C_{q}H_{2q+1})H—O)ᵣH in particular PEG-45/Dodecyl Glycol Copolymer.
(10) Compounds of the type:
CₙH₂ₙ₊₁—(O—CH₂—CH₂)o-O—CH₂—C(CₚH₂ₚ₊1)HOH in particular Ceteareth-60 Myristyl Glycol.
(11) Polyoxyethylenated glycerols, in particular glycerol oxyethylenated with 26 OE (Glycereth-26).
(12) Alkylpolyethylene glycols of the type CₙH₂ₙ₊₁—(O—CH₂—CH₂)ₒ—OH, in particular chosen from Ceteth-2, Ceteth-10, Ceteth-20, Ceteth-25, Isoceteth-20, Laureth-2, Laureth-3, Laureth-4, Laureth-12, Laureth-23, Oleth-2, Oleth-5, Oleth-10, Oleth-20, Oleth-25, Deceth-3, Deceth-5, Beheneth-10, Steareth-2, Steareth-10, Steareth-20, Steareth-21, Steareth-100, Ceteareth-12, Ceteareth-15, Ceteareth-20, Ceteareth-25, Ceteareth-30, Ceteareth-33, Coceth-7 and Trideceth-12.
(13) Polyoxyethylenated alkylamines of the type CH₃—(CH₂)n-(CH═CH)ₐ—(CH)ₚ—N((CH₂—CH₂—O)H)_{q} ((CH₂—CH₂—O)ᵣH), in particular: PEG-2-Oleamine.
(14) Fatty acid esters of polyethylene glycol of the type CₙH₂ₙ₊₁—(CH═CH₂)o-CₚH₂p-CO—(O—CH₂—CH₂)ₙ—OH or CₙH₂ₙ₁—(CH═CH)ₐ—CₚH₂ₚ—CO—(O—CH₂—CH₂)_{q}—O—CO—CrH₂ᵣ₊₁ or CₙH₂ₙ₊₁—(CH═CH)ₐ—CO—(O—CH₂—CH₂)_{q}—O—CH₂ₙ₊₁ or CₙH₂ₙ₊₁—O—CH (alkyl)-(CH₂)ₚ—(O—CH₂—CH₂)_{q}—O—CO—CᵣH₂ᵣ₊₁
in particular chosen from PEG-6 Isostearate, PEG-6 Stearate, PEG-8 Stearate, PEG-8 Isostearate, PEG-20 Stearate, PEG-30 Stearate, PEG-32 Stearate, PEG-40 Stearate, PEG-75 Stearate, PEG-100 Stearate, PEG-8 Distearate, PEG-150 Distearate, Mereth-3 Myristate, PEG-4 Olivate, Propylene Glycol Ceteth-3 Acetate and PEG-30 Dipolyhydroxystearate.
(15) Polyoxyethylenated carboxylic acids of the type CₙH₂ₙ₊₁—(O—CH₂—CH₂)ₒ—COOH, in particular chosen from PEG-7 Capric Acid, PEG-6 Caprylic Acid, PEG-7 Caprylic Acid, Laureth-5 Carboxylic Acid, Laureth-11 Carboxylic Acid and Laureth-12 Carboxylic Acid.
(16) Polyoxyethylenated alkylglycerides, in particular chosen from PEG-6 Caprylic/Capric Glycerides, PEG-60 Almond Glycerides, PEG-10 Olive Glycerides and PEG-45 Palm Kernel Glycerides,

(17) Polyoxyethylenated alkylglucoses, in particular chosen from Methyl-Gluceth-10 and Methyl-Gluceth-20.
(18) Polyoxyethylenated sugar esters such as PEG-120 Methyl Glucose Dioleate or PEG-20 Methyl Glucose Sesquistearate.
(19) Polyoxyalkylenated alkyl glycol ethers such as PPG-1-PEG-9 Lauryl Glycol Ether.
(20) Polyoxyethylenated or polyglycerolated pentaerythritol esters and ethers, in particular chosen from PEG-150 Pentaerythrityl Tetrastearate.
(21) Polysorbates, in particular chosen from Polysorbate-20, Polysorbate-21, Polysorbate-60, Polysorbate-61, Polysorbate-80 and Polysorbate-85.
(22) Polyoxyethylenated polyamines, in particular PEG-15 Cocopolyamine.
(23) Polyoxyethylenated dihydrocholesteryl esters of structure:

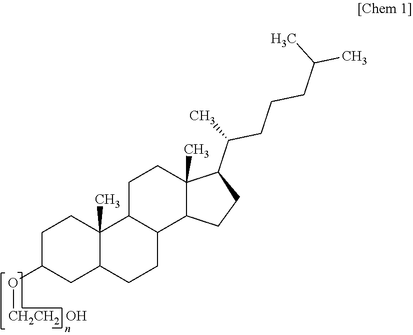

in particular Dihydrocholeth-30.
(24) Polyoxyethylenated ingredients chosen from the mixture of polyoxyethylenated (200 OE) palm glycerides and of polyoxyethylenated (7 OE) coconut kernel oil, PEG-7 Glyceryl Cocoate, PEG-30 Glyceryl Cocoate, PEG-40 Hydrogenated Castor Oil, PEG-60 Hydrogenated Castor Oil, PEG-30 Glyceryl Stearate, PEG-200 Glyceryl Stearate, PEG-20 Glyceryl Triisostearate, PEG-55 Propylene Glycol Oleate, PEG-70 Mango Glycerides, Hydrogenated Palm/Palm Kernel Oil PEG-6 Esters, PEG-200 Hydrogenated Glyceryl Palmitate and PEG-7 Glyceryl Cocoate.
(25) Polyoxyethylenated butters, in particular polyoxyethylenated shea butter.
(26) Polyoxyalkylenated or polyglycerolated silicones, in particular chosen from PEG/PPG-17/18 Dimethicone, PEG/PPG-18/18 Dimethicone, Trideceth-9 PG-Amodimethicone and PEG/PPG-22/24 Dimethicone.
(27) Polyoxyalkylenated or polyglycerolated silanes, in particular chosen from Bis-PEG-18 Methyl Ether Dimethyl Silane and Bis-PEG-18 Methyl Ether Dimethyl Silane.
(28) Polyoxyethylenated or polyglycerolated acrylate copolymers, in particular the copolymer having the INCI name: Acrylate/Palmeth-25 Acrylate Copolymer.
(29) Proteins, in particular modified or unmodified, optionally hydrolysed proteins of plant origin such as silk proteins, keratins, soybean proteins, wheat proteins, corn proteins, lupin proteins, hazelnut proteins, conchiolin proteins, oat proteins, rice proteins and sweet almond proteins.
(30) Polyoxyalkylenated alkanediols such as PEG-8 Caprylyl Glycol.
(31) Polyoxyethylenated rapeseed amides and sterols, in particular chosen from PEG-4 Rapeseed Amide and PEG-5 Rapeseed Sterol.
(32) Polyoxyethylenated lanolins such as Laneth-15.
(33) Polyoxyethylenated fatty acid esters of sorbitol such as PEG-40 Sorbitan Peroleate.
(34) Polyoxyethylenated glycerolated esters such as Glycereth-25 PCA Isostearate.
(35) Polyvinyl alcohols such as those having the following INCI names: Allyl Stearate/Vinyl Alcohol Copolymer, Ethylene/Vinyl Alcohol Copolymer, Polyvinyl Alcohol, Vinyl Alcohol/Crotonates Copolymer, Vinyl Alcohol/Crotonates/Vinyl Neodecanoate Copolymer.
(36) Vinylpyrrolidone copolymers such as those having the following INCI names: Polyvinyl Pyrrolidone/Vinyl Alcohol, Vinyl Pyrrolidone/Eicosene Copolymer, Vinyl Pyrrolidone/Hexadecene Copolymer, Vinyl Pyrrolidone/Dimethylaminopropylacrylamide Acrylates Copolymer, Hydrolysed Wheat Protein/Vinyl Pyrrolidone Crosspolymer, Vinyl Pyrrolidone/Methacrylamide/Vinyl Imidazole Copolymer, Vinyl Pyrrolidone/Acrylates/Lauryl Methacrylate Copolymer, Vinyl Caprolactam/VP/Dimethylaminoethyl Methacrylate Copolymer, Vinyl Pyrrolidone/Dimethylaminoethylmethacrylate Copolymer, Vinyl Pyrrolidone/Polycarbamyl Polyglycol Ester.
(37) Caprolactam polymers and copolymers such as polyvinylcaprolactams, the polymers having the INCI name: Vinyl Caprolactam/Vinyl Pyrrolidone/Dimethylaminoethyl Methacrylate Copolymer.
(38) Polyoxyethylenated amide compounds of the type
CₙH₂ₙ₊₁—(O—CH₂—CH₂) oO—CH₂—CO—NH—(CH₂—CH₂—O)ₚH, in particular Trideceth-2-Carboxamide MEA.
(39) mixtures thereof.

According to a preferential embodiment, the compound(s) Y will be chosen from nonionic compounds.

According to a preferential embodiment, the compound(s) Y will be chosen from pullulans; celluloses such as cetylhydroxyethylcellulose: modified guar gums, in particular hydroxypropyl guar; fatty acid esters of polyglycerols, in particular Polyglyceryl-10 Caprate and Polyglyceryl-10 Laurate; polyethylene glycols such as PEG-180; PEG-40 Hydrogenated Castor Oil; polysorbates, in particular Polysorbate 80; polyoxyalkylenated ester waxes such as polyoxyethylenated (120 OE) jojoba wax; and mixtures thereof.

According to a particular embodiment, the compound(s) Y according to the invention will be present in a content equal or greater than 0.8% by weight, preferably equal or greater than 1.0% by weight, more particularly equal or greater than 2.0% by weight relative to the total weight of the composition (B).

According to a particular embodiment, the compound(s) Y according to the invention will be present in a content from 1.0 to 30.0% by weight and more particularly of from 2.0 to 30% relative to the total weight of the composition (B).

According to a preferential embodiment of the invention, the mole ratio of the reactive hydroxyl groups (OH) of the polyphenol(s) X to the functional groups Gy of compound(s) Y that are reactive with said hydroxyl groups preferentially ranges from ⅓ to 20, more preferentially from ½ to 15 and more particularly from ¾ to 3.

### Two-Stage Process for Coating Keratin Materials

According to a particular embodiment, the invention is a cosmetic process for coating keratin materials, notably for care and/or makeup, more particularly for makeup, comprising at least:
a) a first composition (A) comprising, notably in a physiologically acceptable medium, at least one polyphenol X comprising at least two different phenol groups; and
b) a second composition (B) comprising, notably in a physiologically acceptable medium, at least one compound Y including at least two functional groups Gy, which may be identical or different, which are capable of forming at least two hydrogen bonds with said phenol groups of the polyphenol X;
said compositions (A) and (B) being applied to the keratin materials
i) simultaneously; or ii) in the form of an extemporaneous mixture at the time of use; or iii) successively, irrespective of the order of application.

According to a particular embodiment, the invention relates to a cosmetic kit for coating keratin materials, notably for care and/or makeup, comprising at least:
a) a first composition (A) as defined previously; and
b) a second composition (B) as defined previously; said compositions (A) and (B) being packaged separately.

### Composition (A) Comprising the Polyphenol(s) X

According to a particular embodiment of the invention, composition (A) containing the polyphenol(s) X comprises at least one aqueous phase.

The term “aqueous phase” means a phase comprising water and also optionally all the water-soluble or water-miscible solvents and ingredients (miscibility with water of greater than 50% by weight at 250° C.), for instance lower monoalcohols containing from 2 to 5 carbon atoms such as ethanol or isopropanol, polyols containing from 3 to 8 carbon atoms such as propylene glycol, 1,3-butylene glycol, caprylyl glycol, pentylene glycol, glycerol, and dipropylene glycol; C3-C4 ketones and C2-C4 aldehydes.

The aqueous phase may contain a demineralized water or alternatively a floral water such as cornflower water and/or a mineral water such as Vittel water, Lucas water or La Roche Posay water and/or a spring water.

The amount of water is preferably greater than 30% by weight, or even greater than 40% by weight, more preferentially ranging from 30% to 85% relative to the total weight of composition (A).

The amount of aqueous phase is preferably greater than 10% by weight, or even greater than 20% by weight, more preferentially ranging from 20% to 90%, relative to the total weight of composition (A).

The pH of the aqueous composition (A) is preferably less than 8.0, more preferentially less than 7.0, more particularly ranging from 2 to 6.

According to another particular embodiment of the invention, composition (A) comprises an oily phase.

According to another particular embodiment of the invention, composition (A) is anhydrous.

According to another particular embodiment of the invention, composition (A) is anhydrous and comprises an oily phase.

The term “anhydrous composition” refers to any composition comprising less than 5% by weight of water, or even less than 2% by weight of water, or even less than 1% by weight of water relative to the total weight of the composition, or even is free of water.

The term “oily phase” refers to a phase which is liquid at room temperature and at atmospheric pressure, comprising at least one fatty substance such as oils, waxes or pasty substances and also optionally all the organic solvents and ingredients that are soluble or miscible in said phase.

The oil(s) may be chosen from mineral, animal, plant or synthetic oils; in particular volatile or non-volatile hydrocarbon-based oils and/or silicone oils and/or fluoro oils, and mixtures thereof.

The term “oil” refers to a fatty substance that is liquid at room temperature (250° C.) and atmospheric pressure (760 mmHg, i.e. 105 Pa). The oil may be volatile or non-volatile.

For the purposes of the present invention, the term “silicone oil” means an oil comprising at least one silicon atom, and notably at least one Si—O group, and more particularly an organopolysiloxane.

The term “hydrocarbon-based oil” refers to an oil mainly containing carbon and hydrogen atoms and possibly one or more functions chosen from hydroxyl, ester, ether and carboxylic functions.

For the purposes of the invention, the term “volatile oil” refers to any oil that is capable of evaporating on contact with the skin in less than one hour, at room temperature and atmospheric pressure. The volatile oil is a volatile cosmetic compound, which is liquid at room temperature, notably having a non-zero vapour pressure, at room temperature and atmospheric pressure, notably having a vapour pressure ranging from 2.66 Pa to 40 000 Pa, in particular ranging from 2.66 Pa to 13 000 Pa and more particularly ranging from 2.66 Pa to 1300 Pa.

The term “non-volatile oil” refers to an oil that remains on the skin or the keratin fibre at room temperature and atmospheric pressure for at least several hours, and that notably has a vapour pressure of less than 2.66 Pa, preferably less than 0.13 Pa. By way of example, the vapour pressure may be measured according to the static method or via the effusion method by isothermal gravimetry, depending on the vapour pressure (standard OCDE 104).

### Volatile Hydrocarbon-Based Oils

As examples of volatile hydrocarbon-based oils that may be used in the invention, mention may be made of hydrocarbon-based oils containing from 8 to 16 carbon atoms, and notably C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar® or Permethyl®, branched C₈-C₁₆ esters and isohexyl neopentanoate, and mixtures thereof. Other volatile hydrocarbon-based oils, for instance petroleum distillates, notably those sold under the name Shell Solt by the company Shell, may also be used; volatile linear alkanes, such as those described in patent application DE10 2008 012 457 from the company Cognis.

### Non-Volatile Hydrocarbon-Based Oils

As examples of non-volatile hydrocarbon-based oils that may be used in the invention, mention may be made of:
hydrocarbon-based oils of animal origin, such as perhydrosqualene;
linear or branched hydrocarbons, of mineral or synthetic origin, such as liquid paraffins and derivatives thereof, petroleum jelly, polydecenes, polybutenes or polyisobutenes, which are optionally hydrogenated such as Parleam, or squalane;
synthetic ethers containing from 10 to 40 carbon atoms, such as dicaprylyl ether;
triglycerides consisting of fatty acid esters of glycerol, in particular the fatty acids of which may have chain lengths ranging from C4 to C36, and notably from C₁₈ to C₃₆, these oils possibly being linear or branched, and saturated or unsaturated; these oils may notably be heptanoic or octanoic triglycerides, wheatgerm oil, sunflower oil, grapeseed oil, sesame seed oil (820.6 g/mol), corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea oil; or alternatively caprylic/capric acid triglycerides, for instance those sold by the company Stéarinerie Dubois or those sold under the names Miglyol 810®, 812® and 818® by the company Dynamit Nobel; —linear aliphatic hydrocarbon-based esters of formula RCOOR′ in which RCOO represents a carboxylic acid residue including from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, diisopropyl adipate, heptanoates, and notably isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate and isostearyl behenate; —polyesters obtained by condensation of unsaturated fatty acid dimer and/or trimer and of diol, such as those described in patent application FR 0 853 634, in particular such as of dilinoleic acid and of 1,4-butanediol. Mention may notably be made in this respect of the polymer sold by Biosynthis under the name Viscoplast 14436H® (INCI name: Dilinoleic acid/butanediol copolymer) or copolymers of polyols and of diacid dimers, and esters thereof, such as Hailuscent ISDA®,
dialkyl carbonates, the two alkyl chains possibly being identical or different, such as dicaprylyl carbonate sold under the name Cetiol CC® by Cognis;
linear fatty acid esters with a total carbon number ranging from 35 to 70, for instance pentaerythrityl tetrapelargonate,
aromatic esters such as tridecyl trimellitate C12-C15 alcohol benzoate, the 2-phenylethyl ester of benzoic acid, and butyloctyl salicylate,
esters and polyesters of diol dimer and of monocarboxylic or dicarboxylic acid, such as esters of diol dimer and of fatty acid and esters of diol dimer and of dicarboxylic acid dimer, such as Lusplan DD-DA5© and Lusplan DD-DA7© sold by the company Nippon Fine Chemical and described in patent application US 2004-175 338, the content of which is incorporated into the present application by reference,
fatty alcohols containing from 12 to 26 carbon atoms, for instance octyldodecanol, 2-butyloctanol, 2-hexyldecanol, 2-undecylpentadecanol and oleyl alcohol,
dialkyl carbonates, the two alkyl chains possibly being identical or different, such as dicaprylyl carbonate sold under the name Cetiol CC® by Cognis;
and mixtures thereof.

### Non-Volatile Silicone Oils

Among the non-volatile silicone oils, mention may be made of silicone oils such as non-volatile polydimethylsiloxanes (PDMS); phenyl silicones such as phenyl trimethicones, phenyl dimethicones, diphenyl dimethicones, trimethylpentaphenyltrisiloxanes, tetramethyltetraphenyltrisiloxanes, trimethylsiloxyphenyl dimethicones, diphenylsiloxyphenyl trimethicones, and also mixtures thereof.

### Linear or Cyclic Volatile Silicone Oils

Linear volatile silicone oils that may be mentioned include octamethyltrisiloxane, decamethyltetrasiloxane, dodecamethylpentasiloxane and mixtures thereof.

Cyclic volatile silicone oils that may be mentioned include octamethylcyclotetrasiloxane, decamethylcyclopentasiloxane and dodecamethylcyclohexasiloxane, and mixtures thereof.

Preferably, when composition (A) is anhydrous, the oily phase comprises at least one volatile hydrocarbon-based oil, more preferentially chosen from C₈-C₁₆ isoalkanes of petroleum origin (also known as isoparaffins), for instance isododecane (also known as 2,2,4,4,6-pentamethylheptane), isohexadecane and isodecane, and more particularly isododecane.

When composition (A) is anhydrous, the oily phase concentration of the composition of the invention is preferably greater than 10% by weight, or even greater than 20% by weight, more particularly ranging from 30% to 75% by weight, relative to the total weight of composition (A).

### Composition (B) Comprising the Compound(s) Y

According to a particular embodiment of the invention, composition (B) comprising the compound(s) Y comprises at least one aqueous phase.

The amount of water is preferably greater than 30% by weight, or even greater than 40% by weight, more preferentially greater than 65%, relative to the total weight of composition (B).

The amount of aqueous phase is preferably greater than 10% by weight, or even greater than 20% by weight, more preferentially ranging from 20% to 90%, relative to the total weight of composition (B).

The pH of the aqueous composition (B) is preferably less than 8.0, more preferentially less than 7.0, more particularly ranging from 2 to 6.

According to another particular embodiment, composition (B) may comprise at least one oily phase as defined previously.

According to a particular embodiment of the invention, composition (B) is anhydrous.

According to another particular embodiment, composition (B) is anhydrous and comprises at least one oily phase as defined previously.

Preferably, when composition (B) is anhydrous, the oily phase comprises at least one volatile hydrocarbon-based oil, more preferentially chosen from C₈-C₁₆ isoalkanes of petroleum origin (also known as isoparaffins), for instance isododecane (also known as 2,2,4,4,6-pentamethylheptane), isohexadecane and isodecane, and more particularly isododecane.

When composition (B) is anhydrous, the oily phase concentration of the composition of the invention is preferably greater than 10% by weight, or even greater than 20% by weight, more particularly ranging from 30% to 75% by weight, relative to the total weight of composition (B).

According to a particular form of the invention, composition (A) and composition (B) are mixed extemporaneously at the time of use before being applied to the keratin materials.

Needless to say, a person skilled in the art will take care to select compositions (A) and (B) so that they are compatible and can be mixed and the amounts for obtaining in the mixture obtained the formation of a coating agent by hydrogen bonding interaction of the polyphenol X with the compound Y.

### Dyestuff

According to a particular embodiment of the invention, composition (A) and/or (B) comprises at least one dyestuff, which is synthetic, natural or of natural origin.

The dyestuff may be chosen from coated or uncoated pigments, water-soluble dyes, liposoluble dyes, and mixtures thereof.

### Pigments

The term “pigments” means white or coloured, mineral or organic particles, which are insoluble in an aqueous medium, and which are intended to colour and/or opacify the resulting composition and/or deposit.

According to a particular embodiment, the pigments used according to the invention are chosen from mineral pigments.

The term “mineral pigment” means any pigment that satisfies the definition in Ullmann's encyclopaedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminium powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The size of the pigment that is useful in the context of the present invention is generally greater than 100 nm and may range up to 10 μm, preferably from 200 nm to 5 μm and more preferentially from 300 nm to 1 μm.

According to a particular form of the invention, the pigments have a size characterized by a D[50] greater than 100 nm and possibly ranging up to 10 μm, preferably from 200 nm to 5 μm and more preferentially from 300 nm to 1 μm.

sizes are measured by static light scattering using a commercial MasterSizer 3000® particle size analyser from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicron to multimicron; it allows an “effective” particle diameter to be determined. This theory is notably described in the publication by Van de Hulst, H. C., Light Scattering by Small Particles, Chapters 9 and 10, Wiley, New York, 1957.

D[50] represents the maximum size that 50% by volume of the particles have.

In the context of the present invention, the mineral pigments are more particularly iron oxide and/or titanium dioxide. Examples that may be mentioned more particularly include and titanium dioxides and iron oxide coated with aluminium stearoyl glutamate, sold, for example, under the reference NAI® by the company Miyoshi Kasei.

As mineral pigments that may be used in the invention, mention may also be made of nacres.

The term “nacres” should be understood as meaning coloured particles of any form, which may or may not be iridescent, notably produced by certain molluscs in their shell, or alternatively synthesized, and which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic dyestuffs.

According to a particular embodiment, the pigments used according to the invention are chosen from mineral pigments.

Examples of nacres that may also be mentioned include natural mica covered with titanium oxide, with iron oxide, with natural pigment or with bismuth oxychloride.

The nacres may more particularly have a yellow, pink, red, bronze, orange, brown, gold and/or coppery colour or tint.

Among the pigments that may be used according to the invention, mention may also be made of those having an optical effect different from a simple conventional colouring effect, i.e. a unified and stabilized effect such as produced by conventional dyestuffs, for instance monochromatic pigments. For the purposes of the invention, the term “stabilized” means lacking the effect of variability of the colour with the angle of observation or in response to a temperature change.

For example, this material may be chosen from particles with a metallic tint, goniochromatic colouring agents, diffractive pigments, thermochromic agents, optical brighteners, and also fibres, notably interference fibres. Needless to say, these various materials may be combined in order simultaneously to afford two effects, or even a novel effect in accordance with the invention.

According to a particular embodiment, composition (A) and/or (B) according to the invention comprises at least one uncoated pigment.

According to another particular embodiment, composition (A) and/or (B) according to the invention comprises at least one pigment coated with at least one lipophilic or hydrophobic compound.

This type of pigment is particularly advantageous. Insofar as they are treated with a hydrophobic compound, they show predominant affinity for an oily phase, which can then convey them.

The coating may also comprise at least one additional non-lipophilic compound.

For the purposes of the invention, the “coating” of a pigment according to the invention generally denotes the total or partial surface treatment of the pigment with a surface agent, absorbed, adsorbed or grafted onto said pigment.

The surface-treated pigments may be prepared according to surface treatment techniques of chemical, electronic, mechanochemical or mechanical nature that are well known to a person skilled in the art. Commercial products may also be used.

The surface agent may be absorbed, adsorbed or grafted onto the pigments by evaporation of solvent, chemical reaction and creation of a covalent bond.

According to one variant, the surface treatment consists of coating the pigments.

The coating may represent from 0.1% to 20% by weight and in particular from 0.5% to 5% by weight, relative to the total weight of the coated pigment.

The coating may be produced, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be produced, for example, by chemical reaction of a surface agent with the surface of the solid pigment particles and creation of a covalent bond between the surface agent and the particles. This method is notably described in patent U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited on the surface of the pigments.

When the pigment comprises a lipophilic or hydrophobic coating, it is preferably present in the fatty phase of the composition according to the invention.

According to a particular embodiment of the invention, the pigments may be coated according to the invention with at least one compound chosen from silicone surface agents; fluoro surface agents; fluorosilicone surface agents; metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

According to a particular embodiment of the invention, the pigments may be coated with a hydrophilic compound.

According to a particular embodiment, the dyestuff is an organic pigment, which is synthetic, natural or of natural origin.

The term “organic pigment” refers to any pigment that satisfies the definition in Ullmann's encyclopaedia in the chapter on organic pigments. The organic pigment may notably be chosen from nitroso, nitro, azo, xanthene, (CI uinoline, anthraquinone, phthalocyanine, metal complex type, isoindolinone, isoindoline, (CI uinacridone, perinone, perylene, diketopyrrolopyrrole, thioindigo, dioxazine, triphenylmethane and (CI uinophthalone compounds.

The organic pigment(s) may be chosen, for example, from carmine, carbon black, aniline black, melanin, azo yellow, (CI uinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indolic or phenolic derivatives as described in patent FR 2 679 771.

The pigments may also be in the form of composite pigments as described in patent EP 1184 426. These composite pigments may notably be composed of particles including a mineral core at least partially covered with an organic pigment and at least one binder for fixing the organic pigments to the core.

The pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the name D&C Red 7 (CI 15 850:1).

The pigment(s) are preferably present in composition (A) and/or (B) in contents of less than 60% by weight, or even less than 50% by weight, more particularly ranging from 2% to 50% by weight and even better still from 3% to 45% by weight, relative to the total weight of composition (A) or (B).

According to a particular embodiment of the invention, the dyestuff is a water-soluble dye or a liposoluble dye.

For the purposes of the invention, the term “water-soluble dyestuff” means any natural or synthetic, generally organic compound, which is soluble in an aqueous phase or water-miscible solvents and which is capable of imparting colour.

For the purposes of the invention, the term “liposoluble dyestuff” means any natural or synthetic, generally organic compound, which is soluble in an oily phase or in solvents that are miscible with the oily phase, and which is capable of imparting colour.

As water-soluble dyes that are suitable for use in the invention, mention may notably be made of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5 and FDC Blue 1.

Among the natural water-soluble dyes, mention may be made of anthocyanins.

As liposoluble dyes that are suitable for use in the invention, mention may notably be made of liposoluble dyes, for instance DC Red 17, DC Red 21, DC Red 27, DC Green 6, DC Yellow 11, DC Violet 2, DC Orange 5, Sudan red and Sudan brown.

As illustrations of natural liposoluble dyes, mention may be made particularly of carotenes, for instance β-carotene, α-carotene and lycopene; (CI uinoline yellow; xanthophylls such as astaxanthin, antheraxanthin, citranaxanthin, cryptoxanthin, canthaxanthin, diatomoxanthin, flavoxanthin, fucoxanthin, lutein, rhodoxanthin, rubixanthin, siphonaxanthin, violaxanthin, zeaxanthin; annatto; curcumin; (CI uinizarin (ceres green BB, D&C green No. 6, CI 61565, 1,4-di-p-toluidinoanthraquinone, green No. 202, (CI uinazine green SS) and chlorophylls.

The water-soluble or liposoluble dye(s) are preferably present in composition (A) or (B) in contents of less than 4% by weight, or even less than 2% by weight, more preferentially ranging from 0.01% to 2% by weight and even better still from 0.02% to 1.5% by weight, relative to the total weight of composition (A) or (B).

### Process for Making Up Keratin Materials

The invention relates to a cosmetic process for coating keratin materials, notably for care and/or makeup, more particularly for makeup, which consists in applying to said keratin materials:
a) at least one composition (A) as defined previously and
b) at least one composition (B) as defined previously; composition (A) and/or (B) comprising at least one dyestuff;
said compositions (A) and (B) being applied to the keratin materials i) simultaneously; or ii) in the form of an extemporaneous mixture at the time of use; or iii) successively, irrespective of the order of application.

### Variant 1

According to a first variant, the following are successively applied onto the keratin materials:
a) a first coat of makeup onto the keratin materials with a composition (A) (base coat) as defined previously comprising at least one dyestuff, and then
b) onto the coloured keratin materials, a second post-treatment coat (top coat) with a composition (B) as defined previously.

According to a particular embodiment, said makeup composition (A) is aqueous and the post-treatment composition (B) is aqueous.

According to another particular embodiment, said makeup composition (A) is aqueous and the post-treatment composition (B) is anhydrous.

According to another particular embodiment, said makeup composition (A) is anhydrous and the post-treatment composition (B) is aqueous.

According to another particular embodiment, said makeup composition (A) is anhydrous and the post-treatment composition (B) is anhydrous.

### Variant 2

According to a second variant, the following are successively applied onto the keratin materials:
a) a first coat of makeup (base coat) with a composition (B) as defined previously comprising at least one dyestuff, and then
b) onto the coloured keratin materials, a second post-treatment coat (top coat) with a post-treatment composition (A) as defined previously.

According to a particular embodiment, said makeup composition (B) is aqueous and the post-treatment composition (A) is aqueous.

According to another particular embodiment, said makeup composition (B) is aqueous and the post-treatment composition (A) is anhydrous.

According to another particular embodiment, said makeup composition (B) is anhydrous and the post-treatment composition (A) is aqueous.

According to another particular embodiment, said makeup composition (B) is anhydrous and the post-treatment composition (A) is anhydrous.

### Variant 3

According to a third variant, the following are successively applied onto the keratin materials:
a) a first coat (base coat) for pretreatment of the keratin materials with a composition (A) as defined previously, and then
b) on the preceding coat, a second keratin material makeup coat (top coat) with a composition (B) as defined previously comprising at least one compound Y and at least one dyestuff.

According to a particular embodiment, said pretreatment composition (A) is aqueous and the makeup composition (B) is aqueous.

According to another particular embodiment, said pretreatment composition (A) is aqueous and the makeup composition (B) is anhydrous.

According to another particular embodiment, said pretreatment composition (A) is anhydrous and the makeup composition (B) is aqueous.

According to another particular embodiment, said pretreatment composition (A) is anhydrous and the makeup composition (B) is anhydrous.

### Variant 4

According to a fourth variant, the following are successively applied onto the keratin materials:
a) a first coat (base coat) for pretreatment of the keratin materials with a composition (B) as defined previously, and then
b) on the preceding coat, a second keratin material makeup coat (top coat) with a composition (A) as defined previously comprising at least one dyestuff.

According to a particular embodiment, said pretreatment composition (B) is aqueous and the makeup composition (A) is aqueous.

According to another particular embodiment, said pretreatment composition (B) is aqueous and the makeup composition (A) is anhydrous.

According to another particular embodiment, said pretreatment composition (B) is anhydrous and the makeup composition (A) is aqueous.

According to another particular embodiment, said pretreatment composition (B) is anhydrous and the makeup composition (A) is anhydrous.

Among variants 1 to 4 defined previously, use will preferably be made of variants 1 and 2, in which a first coat of makeup is applied with composition (A) or composition (B) containing said dyestuff(s).

Use will preferably be made as dyestuff of one or more pigments, when composition (A) or the makeup composition (B) comprises an anhydrous support comprising an oily phase.

Use will preferably be made as dyestuff of one or more water-soluble dyes, when composition (A) or the makeup composition (B) comprises an aqueous support.

Among the variants 1 to 4 defined previously, use will more particularly be made of variants 2 and 3 in which the dyestuffs are in composition (B) comprising the compound(s) Y.

According to a particularly preferred embodiment, the following are successively applied onto the keratin materials:
a) a first coat of makeup (base coat) with an anhydrous composition (B) as defined previously comprising at least one dyestuff, preferably a pigment, and then
b) onto the coloured keratin materials, a second post-treatment coat (top coat) with an aqueous composition (A) as defined previously.

### Cosmetic Additives

Compositions (A) and/or (B) of the invention may contain additives that are common in cosmetics. Mention may notably be made of antioxidants, preserving agents, neutralizers, gelling agents or thickeners, surfactants, cosmetic active agents, for instance emollients, moisturizers or vitamins, and mixtures thereof.

### Antioxidants

In particular, the antioxidants are used to prevent the oxidation of the polyphenol X. They may be chosen from ascorbic acid and derivatives thereof, erythorbic acid, sulfites and metabisulfite, and reducing agents of thiol type, in particular cysteine. Mention may also be made of carotenes and lycopenes, which also act as liposoluble dyes.

These additives may be present in compositions (A) and/or (B) in a content ranging from 0.01% to 15.0% relative to the total weight of the composition.

Needless to say, a person skilled in the art will take care to select the optional additional additives and/or the amount thereof such that the advantageous properties of compositions (A) and/or (B) according to the invention are not, or are not substantially, adversely affected by the envisaged addition.

Compositions (A) and/or (B) may be manufactured via the known processes, generally used in the cosmetic field.

Compositions (A) and/or (B) used according to the invention may be care products for keratin materials such as the skin, the area around the eyes, the lips, the hair, the eyelashes, the eyebrows and the nails.

Compositions (A) and/or (B) used according to the invention may be makeup products for keratin materials such as the skin, the area around the eyes, the lips, the eyelashes, the eyebrows and the nails, such as foundations, eyeshadows, lipsticks, mascaras, eyeliners, nail varnishes, primers or finishers.

Compositions (A) and/or (B) used according to the invention may be hybrid products, i.e. products for caring for and making up keratin materials such as the skin, the area around the eyes, the lips, the eyelashes, the eyebrows and the nails, such as foundations, eyeshadows, lipsticks, mascaras, eyeliners and nail varnishes.

### Packaging and Applicators

Compositions (A) and/or (B) according to the invention may each be packaged in a container delimiting at least one compartment that comprises said composition, said container being closed by a closing member.

The container may be in any suitable form. It may notably be in the form of a bottle, a tube, a jar or a case.

The closing member may be in the form of a removable stopper, a lid or a cover, notably of the type including a body fixed to the container and a cap articulated on the body. It may also be in the form of a member for selectively closing the container, notably a pump, a valve or a flap valve.

The container may be combined with an applicator, notably in the form of a brush including an arrangement of bristles maintained by a twisted wire. Such a twisted brush is described notably in patent U.S. Pat. No. 4,887,622. It may also be in the form of a comb including a plurality of application members, obtained notably by moulding. Such combs are described, for example, in patent FR 2 796 529. The applicator may be in the form of a fine brush, as described, for example, in patent FR 2 722 380. The applicator may be in the form of a block of foam or of elastomer. The applicator may be free (sponge) or securely fastened to a rod borne by the closing member, as described, for example, in patent U.S. Pat. No. 5,492,426. The applicator may be securely fastened to the container, as described, for example, in patent FR 2 761 959.

The product may be contained directly in the container, or indirectly.

The closing member may be coupled to the container by screwing. Alternatively, the coupling between the closing member and the container occurs other than by screwing, in particular via a bayonet mechanism, by click-fastening or by gripping. The term “click-fastening” in particular means any system involving the crossing of a bead or cord of material by elastic deformation of a portion, notably of the closing member, followed by return to the elastically unconstrained position of said portion after the bead or cord has been crossed.

The container may be at least partially made of thermoplastic material. Examples of thermoplastic materials that may be mentioned include polypropylene and polyethylene.

The container may have rigid or deformable walls, notably in the form of a tube or a tube bottle.

The container may comprise means intended to bring about or facilitate the dispensing of the composition. By way of example, the container may have deformable walls so as to cause the composition to exit in response to excess pressure inside the container, which excess pressure is brought about by the elastic (or nonelastic) squeezing of the walls of the container

The container may be equipped with a drainer positioned in the vicinity of the opening of the container. Such a drainer makes it possible to wipe the applicator and possibly the rod to which it may be securely fastened. Such a drainer is described, for example, in patent FR 2 792 618.

Throughout the description, including the claims, the term “including a” should be understood as being synonymous with “including at least one”, unless otherwise specified.

The expressions “between . . . and . . . ”, and “ranging from . . . to . . . ” should be understood as meaning limits included, unless otherwise specified. The invention is illustrated in greater detail by the examples and FIGURES presented below. Unless otherwise indicated, the amounts shown are expressed as mass percentages.

### EXAMPLES

a) Two-stage makeup process with 1) application of a first coat (base coat) of composition (A) comprising a polyphenol X (tannic acid) and 2) application of a second coat (top coat) of a composition (B) comprising a compound Y which is capable of interacting with the polyphenol by hydrogen bonding.

The following formulations were prepared:

### Base Coat Compositions

TABLE 1

| | % by weight | |
|---|---|---|
| | | A2 |
| | A1 | outside the |
| Ingredients | (invention) | invention) |
| Tannic acid (from | 0 | |
| Sigma-Aldrich) | | |
| Pigment (black iron | 0 | |
| oxide) CI 77499 | | |
| ineralized water | 0 | 0 |

### Top Coat Compositions

TABLE 2

| | % by weight | | | | | | | | | |
|---|---|---|---|---|---|---|---|---|---|---|
| Compound | B1 | B2 | B3 | B4 | B5 | B6 | B7 | B8 | B9 | B10 |
| Polysorbate-80 | 10 | | | | | | | | | |
| (Tween 80-LQ ® - Croda) | | | | | | | | | | |
| PEG-180 (Polyglycol 8000S ® - | | 10 | | | | | | | | |
| Clariant) | | | | | | | | | | |
| Hydroxypropyl guar | | | 1 | | | | | | | |
| Jaguar HP 105 SGI ® - Solvay) | | | | | | | | | | |
| Pullulan (Cosmetic Grade Pullulan - | | | | 7.1 | | | | | | |
| Hayashibara) | | | | | | | | | | |
| PEG-30 glyceryl stearate | | | | | 10 | | | | | |
| (Tagat S ® - Evonik) | | | | | | | | | | |
| Polyglyceryl-10 laurate | | | | | | 9 | | | | |
| (Dermofeel G10L ® from Dr Straetmans) | | | | | | | | | | |
| Glycereth-26 (Liponic EG-1 ® - Vantage | | | | | | | 10 | | | |
| Specialty Chemicals) | | | | | | | | | | |
| PEG-60 hydrogenated castor oil | | | | | | | | 10 | | |
| (Eumulgin CO 60 ® - BASF) | | | | | | | | | | |
| Cetylhydroxyethyl-cellulose | | | | | | | | | 1 | |
| (Natrosol Plus 330 CS ® - Ashland) | | | | | | | | | | |
| Polyvinyl alcohol | | | | | | | | | | 10 |
| (Selvol Ultalux FF ® - | | | | | | | | | | |
| Sekisui Specialty Chemicals) | | | | | | | | | | |
| Demineralized water | 90 | 90 | 99 | 92.9 | 80 | 91 | 90 | 90 | 99 | 90 |

### Application

Transparent PET plates with a side length of 6 cm were cut.

An adhesive disc (Monaderm® ref. PA22/36 double-sided disc, diameter 22/36), the inner circle of which was 22 mm in diameter, was applied, making it possible to control and to delimit the application area. The same amount of product per unit area was thus applied. 0.1 g of the above formulations was then applied to the PET support as a superposition of 1) base coat and 2) top coat without mixing, allowing the base coat to dry before applying the top coat.

### Persistence Test

The optical transmittance of the deposits thus obtained was measured using a Haze Guard® i machine (Byk). The deposits were then immersed in 50 ml of demineralized water with stirring for 1 minute. After drying, the optical transmittance of the resistant deposits was measured under the same conditions. The change in transmittance was expressed as a percentage increase relative to the transmittance measured before immersion. The higher this increase, the lower the resistance of the deposit to water. The results are collated in the table below:

TABLE 3

| | Base | Top | | | % |
|---|---|---|---|---|---|
| Process | coat | coat | T_{before immersion} | T_{after immersion} | Variation |
| Outside | A1 | — | 3.50 | 77.90 | 2126 |
| the | A2 | B1 | 1.30 | 88.90 | 6738 |
| invention | A2 | B2 | 8.12 | 55.80 | 587 |
| | A2 | B3 | 7.26 | 75.80 | 944 |
| | A2 | B4 | 6.93 | 82.90 | 1096 |
| | A2 | B5 | 9.26 | 40.50 | 337 |
| | A2 | B6 | 4.92 | 52.70 | 971 |
| | A2 | B7 | 4.27 | 14.60 | 242 |
| | A2 | B8 | 2.83 | 30.20 | 967 |
| | A2 | B9 | 3.15 | 3.97 | 26 |
| | A2 | B10 | 2.12 | 40.70 | 1820 |
| Invention | A1 | B1 | 1.20 | 1.72 | 43 |
| | A1 | B2 | 8.34 | 17.00 | 104 |
| | A1 | B3 | 7.77 | 17.40 | 124 |
| | A1 | B4 | 7.01 | 27.30 | 289 |
| | A1 | B5 | 9.26 | 14.00 | 51 |
| | A1 | B6 | 4.64 | 4.97 | 7 |
| | A1 | B7 | 4.54 | 5.12 | 13 |
| | A1 | B8 | 2.94 | 3.39 | 15 |
| | A1 | B9 | 3.04 | 3.11 | 2 |
| | A1 | B10 | 2.15 | 2.84 | 32 |

In contrast with the two-stage makeup processes outside the invention using the polyphenol tannic acid alone or the hydrogen bonding acceptor compound Y alone, the two-stage makeup processes according to the invention using the superposition of a first coat with tannic acid and of a second coat with the hydrogen bonding acceptor compound Y led to a very marked improvement in the water resistance.

1. Cosmetic process for coating keratin material, consists in comprising applying to said keratin material:
a) at least one composition (A) comprising, in a physiologically acceptable medium, at least one polyphenol X comprising at least two different phenol groups; and
b) at least one composition (B) comprising, in a physiologically acceptable medium, at least one compound Y comprising at least two functional groups, which may be identical or different, which are capable of forming at least two hydrogen bonds with said phenol groups of the polyphenol X.

2. Process according to claim 1, in which the polyphenol X is chosen from catechin tannins.

3. Process according to claim 1, in which the polyphenol X is epigallocatechin.

4. Process according to claim 1, in which the polyphenol X is a procyanidin or a mixture of procyanidins.

5. Process according to claim 1, in which the polyphenol X is tannic acid.

6. Process according to claim 1, in which the polyphenol(s) X is present in composition (A) in a content equal or greater than 0.8% by weight relative to the total weight of the composition (A).

7. Process according to claim 1, in which the polyphenol(s) X is present in composition (A) in a content from 1.0 to 30.0% by weight relative to the total weight of the composition (A).

8. Process according to claim 1, in which the compound(s) Y, in the medium of the composition containing it (them), does (do) not contain any anionic group in its(their) structure.

9. Process according to claim 1, in which compound Y comprises at least two functional groups Gy, which may be identical or different, chosen from hydroxyl, acid anhydride, amine, amide, carbamate, urethane, carbamide, urea, thiol, glyceryl, acrylate, acrylamide, vinylpyrrolidone, vinyl alcohol, vinylamine, vinylformamide, and mixtures thereof.

10. Process according to claim 1, the compound(s) Y is (are) different from the sugars obtained from fruits or vegetables.

11. Process according to claim 1, in which the compound(s) Y are nonionic.

12. Process according to claim 1, in which the compound(s) Y is (are) present in composition (B) in a content equal or greater than 0.8% by weight relative to the total weight of the composition (B).

13. Process according to claim 1, in which the compound(s) Y is (are) present in composition (B) in a content from 1.0 to 30.0% by weight relative to the total weight of the composition (B).

14. Process according to claim 1, in which the mole ratio of the reactive hydroxyl groups (OH) of the polyphenol(s) X to the reactive functional group(s) Gy of the compound(s) Y ranges from ⅓ to 20.

15. Process according to claim 1, in which composition (A) and/or composition (B) comprises at least one aqueous phase.

16. Process according to claim 15, in which water is present in a concentration of greater than 30% by weight, relative to the total weight of composition (A) and/or composition (B).

17. Process according to claim 15, in which the pH of composition (A) and/or of composition (B) is less than 8.0.

18. Process according to claim 1, in which composition (A) and/or composition (B) comprises an oily phase.

19. Process according to claim 1, in which composition (A) and/or composition (B) is anhydrous.

20. Process according to claim 18, in which the oily phase concentration is greater than 10% by weight relative to the total weight of composition (A) and/or of composition (B).

21. Cosmetic kit for coating keratin material comprising:
a) a first composition (A) as defined in claim 1; and
b) a second composition (B) as defined in claim 1, said compositions (A) and (B) being packaged separately.

22. Process for making up keratin material according to claim 1 in which composition (A) and/or composition (B) comprises at least one dyestuff which is synthetic, natural or of natural origin.

23. Process for making up keratin materials according to claim 22, in which the following are successively applied:
a) a first coat of makeup on the keratin material with a composition (A) according to claim 22, comprising at least one dyestuff, and then
b) on the first coat of makeup a coloured second coat with a composition (B).

24. Process for making up keratin material according to claim 22, in which the following are successively applied:
a) a first coat of makeup on the keratin material with a composition (B according to claim 22 comprising at least one dyestuff, and then
b) on the first coat of makeup a second coat with a composition (A).

25. Process for making up keratin material according to claim 22, in which the following are successively applied:
a) a first coat on the keratin material for pretreatment of the keratin material with a composition (A), and then
b) on the first, a second coat for making up the keratin material with a composition (B) according to claim 22 comprising at least one dyestuff.

26. Process for making up keratin material according to claim 22, in which the following are successively applied:
a) a first coat on the keratin material for pretreatment of the keratin material with a composition (B), and then
b) on the first coat, a second coat for making up the keratin material with a composition (A) according to claim 22 comprising at least one dyestuff.

27. Process for making up keratin material according to claim 22, in which the following are successively applied:
a) a first coat of makeup on the keratin material with an anhydrous composition (B) according to claim 22 comprising at least one dyestuff, and then
b) on the first coat of makeup, a second coat with an aqueous composition (A).
